(19) United States
(12) Patent Application Publication
(10) Pub. No.: US 2007/0282207 A1
(43) Pub. Date: Dec. 6, 2007

(54) Apparatus for displaying blood pressure and a method thereof

(76) Inventors: Kun-Sung Chen, San-Chung (TW); Hsing Ou Yang, San-Chung (TW); Hsin-Lung Tu, San-Chung (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/445,171

(22) Filed: Jun. 2, 2006

(51) Int. Cl. A61B 5/02

(52) U.S. Cl. 600/485

(57) ABSTRACT

An apparatus for displaying the blood pressure and a method thereof are disclosed. The apparatus for displaying the blood pressure use a blood-measuring unit to receive an analog blood pressure signal. An analog-to-digital converter converts the analog blood pressure signal into a digital blood pressure signal. An LCD driving unit couples to the analog-to-digital converter. The LCD driving unit receives the digital blood pressure signal so as to output an LCD driving voltage. An LCD unit couples to the LCD driving unit and is controlled by the LCD driving voltage to display a color that corresponds to the LCD driving voltage.

### BACKGROUND OF THE INVENTION

1. Field of the Invention

The present invention relates to an apparatus for displaying blood pressure and a method thereof. In particular, this invention relates to an apparatus for displaying the blood pressure and a method thereof that uses an emitting light of an LCD unit to display the status of measured blood pressure.

2. Description of the Related Art

Blood pressure is the pressure generated in blood vessels when blood circulates through a body. When the heart is in systole, blood is transmitted to the entire body via aortas. The generated pressure is named systolic pressure. When the heart is in diastole, blood in the body flows back to the heart. The generated pressure is named diastolic pressure. When blood pressure is abnormal, the blood pressure is an indictor of diseases, such as apoplexy, heart disease, or uremia, etc. Some patients don't feel uncomfortable when their blood pressure is abnormal. Therefore, measuring the blood pressure gives us an insight into our physical condition.

Blood pressure usually is measured by pressing blood vessels via a tourniquet and stethoscope. Blood-measuring instruments can be divided into two types—mercury sphygmomanometer and auscultation sphygmomanometer. Their common point is the use of a tourniquet filled with air to press the artery so as to stop the flood. In this time, the artery under the tourniquet does not generate a pulse. Then, the air in the tourniquet is relaxed to allow the artery to generate pulses again and the pulse is detected. When the first sound of the Korotoff Sound is detected, the measured value in the pressure gauge is systolic pressure. Then, the air in the tourniquet continues to be released. The channel in the blood vessel becomes larger and the pulsing time of blood in the blood vessel becomes longer. Until the pressure in the tourniquet is less than a predetermined value, the pulsing effect of blood becomes less. When the Korotoff Sound becomes passivated, the final Korotoff Sound means diastolic pressure has been found. The mercury sphygmomanometer has a mercury display for displaying the measured blood pressure. When the mercury column of the mercury display is faulty, the display of the mercury sphygmomanometer doesn't function. Therefore, the auscultation sphygmomanometer was developed. The auscultation sphygmomanometer displays the measured blood pressure on the LCD. It is convenient for the user to obtain their blood pressure. However, the user has to refer to a normal blood pressure table to obtain the status of the blood pressure. The user cannot directly obtain their own blood pressure status. It is inconvenient.

### SUMMARY OF THE INVENTION

One particular aspect of the present invention is to provide an apparatus for displaying the blood pressure and a method thereof. The present invention uses the colors emitting from an LCD unit to display the status of the measured blood pressure. After the blood pressure is measured, the user obtains the blood pressure status, without having to refer to a normal blood table.

The apparatus for displaying the blood pressure includes a blood-measuring unit, an analog-to-digital converter, an LCD driving unit and an LCD unit. The blood-measuring unit receives an analog blood pressure signal. The analog-to-digital converter couples to the blood-measuring unit. The analog-to-digital converter converts the analog blood pressure signal into a digital blood pressure signal. The LCD driving unit couples to the analog-to-digital converter. The LCD driving unit receives the digital blood pressure signal so as to output an LCD driving voltage. The LCD unit couples to the LCD driving unit and is controlled by the LCD driving voltage to display a color that corresponds to the LCD driving voltage.

The method for displaying blood pressure includes obtaining an analog blood pressure signal, amplifying the analog blood pressure signal, converting the analog blood pressure signal into a digital blood pressure signal, generating an LCD driving voltage according to the digital blood pressure signal, and displaying a light with a color that corresponds to the LCD driving voltage.

For further understanding of the invention, reference is made to the following detailed description illustrating the embodiments and examples of the invention. The description is only for illustrating the invention and is not intended to be considered limiting of the scope of the claim.

### BRIEF DESCRIPTION OF THE DRAWINGS

The drawings included herein provide a further understanding of the invention. A brief introduction of the drawings is as follows:

FIG. 1 is a schematic diagram of the appearance of the apparatus for displaying the blood pressure of the present invention;

FIG. 2 is a block diagram of the apparatus for displaying the blood pressure of the present invention; and

FIG. 3 is a flow chart of the method for displaying the blood pressure of the present invention.

### DETAILED DESCRIPTION OF THE PREFERRED EMBODIMENTS

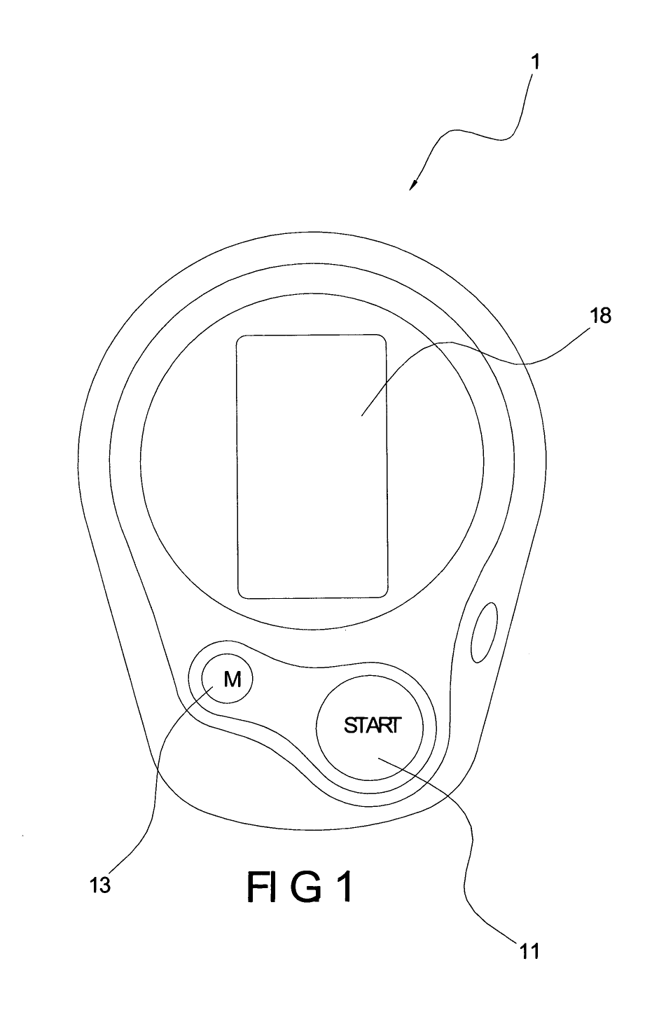

Reference is made to FIG. 1, which shows a schematic diagram of the appearance of the apparatus for displaying the blood pressure of the present invention. On the apparatus for displaying the blood pressure 1, there is an LCD unit 18, a blood pressure measuring button 11, and a blood pressure recording button 13. There are other units in the apparatus for displaying the blood pressure 1. The apparatus for displaying the blood pressure 1 obtains an analog blood pressure signal via a blood pressure measuring unit (including an air tube connected with a tourniquet, not shown in the figure). The user presses the blood pressure measuring button 11 to measure the blood pressure and presses the blood pressure recording button 13 to store the blood pressure when the blood pressure is measured.

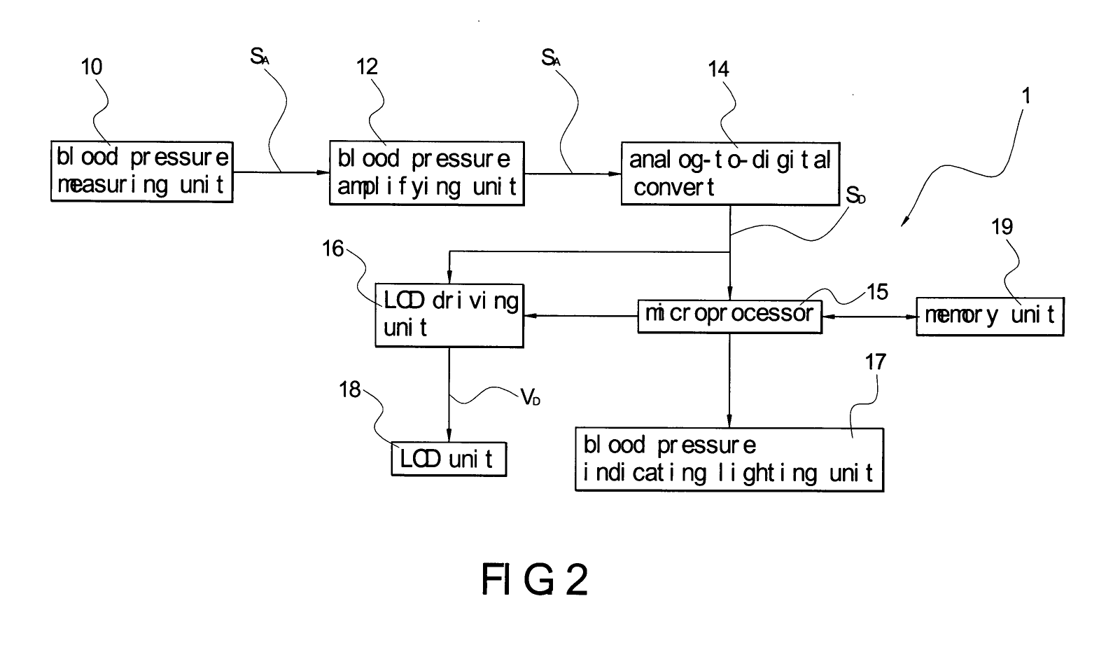

Reference is made to FIG. 2, which shows a block diagram of the apparatus for displaying the blood pressure of the present invention. The apparatus for displaying the blood pressure 1 includes a blood-measuring unit 10, a blood pressure amplifying unit 12, an analog-to-digital converter 14, an LCD driving unit 16, and an LCD unit 18. The apparatus for displaying the blood pressure 1 uses the blood-measuring unit 10 that is composed of an air tube (not shown in the figure) connected with a tourniquet (not shown in the figure) to receive an analog blood pressure signal SA. The analog blood pressure signal SA is amplified by the blood pressure amplifying unit 12 that is connected with the blood-measuring unit 10 and is then transmitted to the analog-to-digital converter 14 that couples to the blood pressure amplifying unit 12. The analog-to-digital converter 14 converts the analog blood pressure signal SA into a digital blood pressure signal SD. The LCD driving unit 16 couples to the analog-to-digital converter 14. The LCD driving unit 16 receives the digital blood pressure signal SD so as to output an LCD driving voltage VD. The LCD unit 18 couples to the LCD driving unit 16 and is controlled by the LCD driving voltage VD to display a color that corresponds to the LCD driving voltage VD. The LCD unit 18 can display a backlight with various colors, including red, yellow, and green, etc.

Reference is made to FIG. 2. The apparatus for displaying the blood pressure 1 further includes a microprocessor unit 15. The microprocessor unit 15 couples to the analog-to-digital converter 14 and the LCD driving unit 16. The microprocessor unit 15 receives the digital blood pressure signal SD so as to control the LCD driving unit 16 to drive the LCD unit 18, and displays the digital blood pressure signal SD on the LCD unit 18. The microprocessor unit 15 further couples to a blood pressure indicating lighting unit 17. The microprocessor unit 15 controls the blood pressure indicating lighting unit 17 to shine according to the digital blood pressure signal SD. The blood pressure indicating lighting unit 17 is composed of at least one LED. The color of the LED can be red, yellow, or green. The microprocessor unit 15 further couples to a memory unit 19. The memory unit 19 is used for storing the digital blood pressure signal SD.

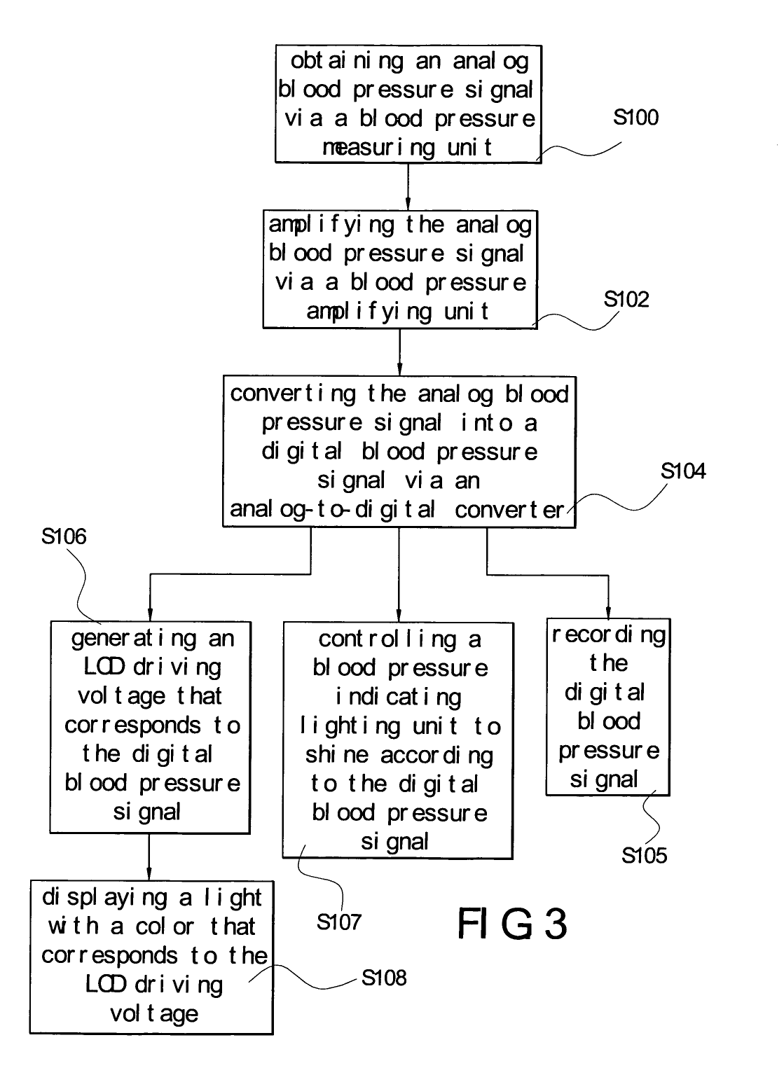

Reference is made to FIG. 2 and FIG. 3. FIG. 3 shows a flow chart of the method for displaying the blood pressure of the present invention. When the user uses the apparatus for displaying the blood pressure to measure the blood measure, the user uses the blood-measuring unit 10 that is composed of an air tube (not shown in the figure) connected with a tourniquet (not shown in the figure) to obtain an analog blood pressure signal SA (S100). The measured analog blood pressure signal SA is amplified by the blood pressure amplifying unit 12 (S102) and is then transmitted to the analog-to-digital converter 14. The analog-to-digital converter 14 converts the analog blood pressure signal SA into a digital blood pressure signal SD (S104) and transmits the digital blood pressure signal SD to the LCD driving unit 16 and the microprocessor 15. The LCD driving unit 16 generates and outputs the corresponding LCD driving voltage VD according to the digital blood pressure signal SD (S 106). The microprocessor unit 15 controls the blood pressure indicating lighting unit 17 to shine (S107) according to the digital blood pressure signal SD, and stores the digital blood pressure signal SD in the memory unit (S105). The LCD driving voltage VD is used for controlling the LCD unit 18 to display a light with a corresponding color (S108).

The present invention provides an apparatus for displaying the blood pressure and a method thereof. The present invention uses the colors emitting from the LCD unit 18 to show the status of the measured blood pressure. After the blood pressure is measured, the user obtains the blood pressure status and records the measured blood pressure. The user easily obtains their health condition without having to refer to a normal blood table.

The description above only illustrates specific embodiments and examples of the invention. The invention should therefore cover various modifications and variations made to the herein-described structure and operations of the invention, provided they fall within the scope of the invention as defined in the following appended claims.

What is claimed is:

1. An apparatus for displaying the blood pressure, comprising:
a blood-measuring unit receiving an analog blood pressure signal;
an analog-to-digital converter coupled to the blood-measuring unit, wherein the analog-to-digital converter converts the analog blood pressure signal into a digital blood pressure signal;
an LCD driving unit coupled to the analog-to-digital converter, wherein the LCD driving unit receives the digital blood pressure signal so as to output an LCD driving voltage; and
an LCD unit coupled to the LCD driving unit and controlled by the LCD driving voltage to display a color that corresponds to the LCD driving voltage.

2. The apparatus for displaying the blood pressure as claimed in claim 1, wherein the LCD unit displays a backlight with a color, including red, yellow, or green.

3. The apparatus for displaying the blood pressure as claimed in claim 1, further comprising a blood pressure amplifying unit coupled to the blood-measuring unit and analog-to-digital converter, wherein the blood pressure amplifying unit amplifies the analog blood pressure signal and then transmits the analog blood pressure signal to the analog-to-digital converter.

4. The apparatus for displaying the blood pressure as claimed in claim 3, further comprising a microprocessor unit coupled to the analog-to-digital converter and the LCD driving unit, wherein the microprocessor unit receives the digital blood pressure signal so as to control the LCD driving unit to drive the LCD unit, and displays the digital blood pressure signal on the LCD unit.

5. The apparatus for displaying the blood pressure as claimed in claim 4, wherein the microprocessor further couples to a blood pressure indicating lighting unit, and the microprocessor unit controls the blood pressure indicating lighting unit to shine according to the digital blood pressure signal.

6. The apparatus for displaying the blood pressure as claimed in claim 5, wherein the blood pressure indicating lighting unit is composed of at least one LED.

7. The apparatus for displaying the blood pressure as claimed in claim 6, wherein the color of the LED is red, yellow, green, or white.

8. The apparatus for displaying the blood pressure as claimed in claim 4, wherein the microprocessor further couples to a memory unit for storing the digital blood pressure signal.

9. A method for displaying the blood pressure, comprising:
obtaining an analog blood pressure signal;
amplifying the analog blood pressure signal;
converting the analog blood pressure signal into a digital blood pressure signal;
generating an LCD driving voltage that corresponds to the digital blood pressure signal according to the digital blood pressure signal; and
displaying a light with a color that corresponds to the LCD driving voltage.

10. The method for displaying the blood pressure as claimed in claim 9, wherein after the step of converting the analog blood pressure signal into a digital blood pressure signal, the method further comprises a step of controlling a blood pressure indicating lighting unit to shine according to the digital blood pressure signal.

11. The method for displaying the blood pressure as claimed in claim 9, wherein after the step of converting the analog blood pressure signal into a digital blood pressure signal, the method further comprises a step of recording the digital blood pressure signal.
